(19) United States
(12) Patent Application Publication
(10) Pub. No.: US 2015/0335571 A1
(43) Pub. Date: Nov. 26, 2015

(54) Pseudobezoar-Based Intraluminal Gastrointestinal Transplant

(71) Applicant: EATLITTLE INC., Calgary, (CA)

(72) Inventors: Orly Yadid-Pecht, Calgary (CA); Martin Mintchev, Calgary (CA)

(21) Appl. No.: 14/759,011

(22) PCT Filed: Jan. 2, 2014
(86) PCT No.: PCT/CA2014/050001

§ 371 Date: Jul. 2, 2015

Related U.S. Application Data
(60) Provisional application No. 61/848,343, filed on Jan. 2, 2013.

(51) Int. Cl. A61K 9/00; A61M 29/02; A61M 31/00

(52) U.S. Cl. CPC A61K 9/0053; A61M 31/002; A61M 29/02; A61M 2202/068; A61M 2210/1042; A61M 2205/3507; A61M 2205/33

(57) ABSTRACT

The present disclosure describes an orally administrable implement comprising expandable material designed specifically to swell in a targeted gastrointestinal (GI) organ of a mammai, including human to form a pseudobezoar. The said pseudobezoar is integrated with a gastrointestinal transplant, whether synthetic or obtained from a donor. Upon swelling of the pseudobezoar in targeted organ GI organ, the gastrointestinal transplant integrated with it is delivered to the targeted gastrointestinal organ in a timed and controllable fashion. In one design, a feedback mechanism reporting on the delivery timing, pattern, and details can be included.

### 1. FIELD OF THE INVENTION

The present invention relates to the field of ingestible medical devices, and, more specifically, to an orally administrable implement comprising expandable structure designed specifically to swell in a targeted gastrointestinal (GI) organ of a mammal, including human to form at least one pseudobezoar. The pseudobezoar contains at least one gastrointestinal transplant containing healthy content from a targeted donor gastrointestinal organ to be administered to a targeted recipient gastrointestinal organ, so that upon swelling of the pseudobezoar in the targeted gastrointestinal organ, the transplant is utilized to modify the microbiotic, bacterial, enzymatic, or any other environment of the said organ. After the transplant is transferred to the targeted GI organ, the pseudobezoar is expelled from the body in a natural way, either in its entirety or after change in dimensions. A safety mechanism for the controlled change in dimensions of the pseudobezoars in cases of possible obstruction or for any other reason is also described.

In the context of the present invention, the term gastrointestinal transplant is utilized, but the synonymous terms are gastrointestinal bacteriotherapy, or microbiota transplantation. These terms are considered to be completely equivalent and interchangeable.

### 2. AIM OF DISCLOSURE

The aim of this disclosure is to offer a technology of creating at least one controllable, organ-targeting gastrointestinal pseudobezoar with the purpose (a) to deliver at least one gastrointestinal transplant to a targeted gastrointestinal organ; (b) to have the means of controlling the said delivery of at least one gastrointestinal transplant in terms of time and duration; and (c) to have the means for the entire implement to harmlessly exit the gastrointestinal system without creating any obstruction or unwanted health effects.

### 3. BACKGROUND OF THE INVENTION

In the present disclosure we will discuss the colon as the targeted GI organ of interest. However, the described methodology is applicable to all other organs in the GI tract, e.g. the throat, the esophagus, the stomach, the duodenum and the small intestine. Each component of the invention, namely, (a) pseudobezoars; (b) fecal transplants; (c) organ-targeting ingestible shell covers; (d) integration of the pseudobezoars, and the fecal transplant; (e) timing and delivery control of the at least one transplant; (f) feedback mechanism monitoring and reporting the said fecal implant delivery; and (g) controlled change in dimensions of the entire implement, will be separately discussed.

### 3.1. Pseudobezoars

Recently proposed pseudobezoar technology has been suggested for the treatment of obesity and for controlled drug delivery in the body (see e.g. US Patent Application Publication Nos. 2010/0215732, 2010/0145316, 200910035367; which are incorporated herein by reference, in their entirety). In the present application we suggest to utilize these GI retaining devices as platforms for (a) delivery of at least one gastrointestinal transplant from selected gastrointestinal organ donor(s) to a targeted GI organ recipient; (b) timing the transplant delivery procedure; and (c) safe exit of the pseudobezoar-based GI transplant delivery platform from the body of the recipient after the delivery of the transplant or if such delivery becomes unwanted or unsafe.

### 3.2. Fecal Transplants and Their Applications

Fecal transplants are new therapy methods involving the administration of feces from healthy subjects to the colons of patients suffering from a variety of disorders, such as infection with clostridium difficile bacteria [see e.g. Gough, E., Shaikh, H., & Manges, A. R. (2011). Systematic review of intestinal microbiota transplantation (fecal bacteriotherapy) for recurrent Clostridium difficile infection. Clinical infectious diseases, 53(10), 994-1002.], various forms of colitis [see e.g. Borody, T. J., & Khoruts, A. (2011). Fecal microbiota transplantation and emerging applications. Nature Reviews Gastroenterology and Hepatology, 9(2), 88-96.], obesity [see e.g. Tilg, H., & Kaser, A. (2011). Gut microbiome, obesity, and metabolic dysfunction. The Journal of clinical investigation, 121(6), 2126.], etc.

Fecal transplantation has been the subject of a recent US patent application publication (2011/0081320, Apr. 7, 2010; incorporated herein by reference in entirety), which discusses the treatment of the following autoimmune disorders: Multiple Sclerosis, Ulcerative Colitis, Lupus Erythematosus, Myasthenia Gravis, Uveoretinitis, Arthritis, Autoimmune Diabetes, and Autoimmune Neuritis, for example, Guillain Barre Syndrome. However, it is envisioned in this application as being administered via enema. Another way of administering fecal transplantation is via colonoscopy [e.g. Yoon, S. S., & Brandt, L. J. (2010). Treatment of refractory/recurrent C. difficile-associated disease by donated stool transplanted via colonoscopy: a case series of 12 patients. Journal of clinical gastroenterology, 44(8), 562-566].

All these techniques for administering fecal transplants are invasive, and therefore, uncomfortable for patients. Moreover, these procedures cannot be administered very often, and the positive clinical effect of the fecal transplant cannot be regularly maintained.

In other US patent applications, oral administration of specific bacteria is envisioned, targeting various gastrointestinal organs. However, they do not envision the pseudobezoar-based delivery, with the help of which the dynamics of the delivery and its sustainability in the targeted organ can be controlled and a feedback of the said delivery and its dynamics can be obtained. Examples of known prior art applications are listed below according to their publication numbers:
20120282675 NOVEL LACTOBACILLUS PLANTARUM AND COMPOSITION CONTAINING SAME
20120277293 Immunostimulatory Method
20120196352 NOVEL LACTOBACILLUS PLANTARUM AND COMPOSITION COMPRISING SAME
20120172420 Method for Modulating Responsiveness to Steroids
20120039860 COMPOSITIONS AND METHODS FOR IMPROVED ORAL HEALTH
20120020950 ANTIBACTERIAL COMPOUNDS
20110312061 NOVEL LACTOBACILLUS PLANTARUM AND COMPOSITION CONTAINING THE SAME
20110206650 Probiotic Formulations
20110081328 Use of selected lactic acid bacteria for reducing atherosclerosis
20110081320 Treatment/Cure of Autoimmune Disease
20110020265 USE OF OLIGOMERS OF LACTIC ACID IN THE TREATMENT OF GYNAECOLOGICAL DISORDERS
20100234449 Immunostimulatory Method
20100004319 Composition and Method for the Prevention, Treatment and/or Alleviation of an Inflammatory Disease
20090175911 Bacterial Production of Carotenoids
20090136454 Anti-inflammatory activity from lactic acid bacteria
20090123467 Polypeptide-Nucleic Acid Conjugate for Immunoprophylaxis or Immunotherapy for Neoplastic or Infectious Disorders
20080318885 Method for Modulating Responsiveness to Steroids
20080268006 Probiotic Lactobacillus Strains for Improved Vaginal Health
20080254011 Use of selected lactic acid bacteria for reducing atherosclerosis
20070123460 Probiotic compounds from Lactobacillus GG and uses therefor
20040241149 Use of unmethylatd cpg
20040208863 Anti-inflammatory activity from lactic acid bacteria
20040106185 Oral bacteriotherapy compositions and methods
20040028689 Probiotic recolonisation therapy
20030147923 Composition & corresponding method of using spores of bacillus subtilis to stimulate and/or enhance immune response in mammals
20020131996 Methods and compositions for blocking microbial adherence to eukaryotic cells

### 4. SUMMARY OF THE INVENTION

The subject of the present invention is to suggest a new method and apparatus for administering GI transplants in a non-invasive way via an ingestible pseudobezoar, initially contained within a specifically-coated standard swallowable shell cover (capsule), which upon reaching the targeted GI organ disintegrates, creating conditions for the pseudobezoar contained in it to rapidly swell. The swelling material in the pseudobezoar, or the pseudobezoar container, or both, can be impregnated with a GI transplant obtained from a healthy donor, which transplant is then delivered to the targeted GI organ of the patient. Alternatively, or concurrently, the pseudobezoar can contain a controllable embedded container, filled with the gastrointestinal transplant, which can be released at a predetermined time, either completely, or gradually, or intermittently. The prolonged presence of the said pseudobezoar in the targeted GI organ ensures the optimal transfer of the GI transplant to the recipient. Prolonged presence of the pseudobezoar in a targeted GI organ is related to the degree of its swelling. For example, if the targeted organ is the stomach, and the pseudobezoar swells to more than 2 cm in diameter with compliance characteristics not permitting reduction in size below this value upon the exertion of force on it equal to, or less than 2N, the said pseudobezoar will be retained in the stomach for as long as it is intact. In a longitudinal organ like the colon, longer retainment can be ensured if the pseudobezoar swells to such extent that it touches the walls of the organ. The higher the friction between the colonic walls and the swollen pseudobezoar, the slower the transit time of the pseudobezoar in the colon.

Regular intake of pseudobezoars containing GI transplant or transplants can maintain the desired therapeutic and clinical effect without the need for repetitive enemas or colonoscopies (in the case of fecal transplants). At the same time, it would open the possibility to apply the new GI transplant technique to other GI organs as well. The easy, non-invasive administration of the GI transplant-containing pseudobezoar via swallowable capsule intake, can enable continuous treatment as required, even a few times a day.

### 4.1. Organ-Targeting Ingestible Shell Covers

Gastrointestinal organ targeting ingestible shell covers are well known in the art. For example, colon-targeting shell covers have been developed utilizing various principles (see e.g. Kosaraju, S. L. (2005). Colon targeted delivery systems: review of polysaccharides for encapsulation and delivery. Critical reviews in food science and nutrition, 45(4), 251-258; Cole, Ewart T., Robert A. Scott, Alyson L. Connor, Ian R. Wilding, Hans-U. Petereit, Carsten Schminke, Thomas Beckert, and Dominique Cadé. “Enteric coated HPMC capsules designed to achieve intestinal targeting.” International journal of pharmaceutics 231, no. 1 (2002): 83-95; Kumar, K. V., Sivakumar, T., & Mani, T. T. (2011). Colon targeting drug delivery system: A review on recent approaches. International Journal Pharm Biomedical Sciences, 2(1), 11-19; Saigal, N., Baboota, S., Ahuja, A., & Ali, J. (2009). Site specific chronotherapeutic drug delivery systems: a patent review. Recent patents on drug delivery & formulation, 3(1), 64-70; Watts, P., & Smith, A. (2005). TARGIT™ technology: coated starch capsules for site-specific drug delivery into the lower gastrointestinal tract. Expert opinion on drug delivery, 2(1), 159-167). Targeting the small intestines is also known in the art (see e.g. Streubel, A., Siepmann, J., & Bodmeier, R. (2006). Drug delivery to the upper small intestine window using gastroretentive technologies. Current opinion in pharmacology, 6(5), 501-508). Gastric-targeting technologies are also known in the art (see e.g. Vardakou, M., A. Mercuri, T. A. Naylor, D. Rizzo, J. M. Butler, P. C. Connolly, M. S. J. Wickham, and R. M. Faulks. “Predicting the human “in vivo” performance of different oral capsule shell types using a novel “in vitro” dynamic gastric model.” International journal of pharmaceutics 419, no. 1 (2011): 192-199). Recent technologies allow even to select the targeted organ (see e.g. Matsusaki, M., & Akashi, M. (2009). Functional multilayered capsules for targeting and local drug delivery. Expert Opinion on Drug Delivery, 6(11), 1207-1217). To summarize, oral targeting shell covers have been known in the art and are commercially available. Possible covers of the capsule can be pH triggered, enzyme triggered or bacteria triggered, or a combination of such. Also, the thickness of the coating and the combination of the different layer materials can control the timing of when the cover will disintegrate in the organ.

### 4.2. Integration of Pseudobezoars and Fecal Transplants

The integration of the at least one pseudobezoar in the implement, and the at least one gastrointestinal transplant can be done in several different ways. Some of them are listed herein in a non-limiting but illustrative fashion.

For example, the fecal transplant can be impregnated to the at least one swellable superabsorbent polymer cluster inside the permeable pseudobezoar container, using a previously described technique of modifying hydrogels to control the release of substance (see e.g. Takemoto, Y., Ajiro, H., Asoh, T. A., & Akashi, M. (2010). Fabrication of Surface-Modified Hydrogels with Polyion Complex for Controlled Release. Chemistry of Materials, 22(9), 2923-2929).

Another approach to integrate the fecal transplant into the implement can be to impregnate the permeable cover of the at least one pseudobezoar container with the at least one fecal transplant, using a previously described technique of modifying the textile to accommodate specific functionality in healing (see e.g. Wollina, U., Heide, M., Müller-Litz, W., Obenauf, D., & Ash, J. (2003). Functional textiles in prevention of chronic wounds, wound healing and tissue engineering. Textiles and the Skin. Curr Probl Dermatol. Basel, Karger, 31, 82-97). In such an application the permeable cover of the pseudobezoar container would be made from biocompatible medical textile.

Yet another possible approach for integrating the at least one fecal transplant into the structure of the implement, without it being limiting whatsoever, would be to include its content in an at least one embedded container residing within the at least one permeable pseudobezoar container. This approach has been previously described (US Patent Application Publication No. 2010/0215732; incorporated herein by reference in its entirety) where a dedicated container is utilized to enable specific controlled release. The container can encapsulate the fecal transplant material and can have different thickness levels or other means of control to time the release of the material within.

To summarize, different ways are possible for achieving the fecal transplant within the container, either by being impregnated in the granules, or by being weaved into the container gauze material, or by being in a dedicated container embedded in the main container, together but separate from the other different granule clusters.

### 4.3. Timing and Delivery Control

The issue of timing and delivery control can also be addressed in widely diverse ways. Several examples are listed here, and the list should not be considered limiting but only illustrative.

One approach for estimating the timing for the delivery of the entire fecal transplant in the cases where the fecal transplant will be impregnated into the at least one swellable cluster contained within the at least one pseudobezoar, or into the permeable lining of the container of the at least one pseudobezoar, is to create a dynamic model of the process of osmosis in the colon with which the fecal transplant will exit the implement and will join the colonic environment (see e.g. Mandal, A. S., Biswas, N., Karim, K. M., Guha, A., Chatterjee, S., Behera, M., & Kuotsu, K. (2010). Drug delivery system based on chronobiology—A review. Journal of Controlled Release, 147(3), 314-325). Obviously, these dynamic models and the estimated delivery time should be verified in a model dynamic colonic environment, which could be either laboratory (see e.g. Oomen, Agnes G., Alfons Hack, Mans Minekus, Evelijn Zeijdner, Christa Cornelis, Greet Schoeters, Willy Verstraete et al. “Comparison of five in vitro digestion models to study the bioaccessibility of soil contaminants.” Environmental science & technology 36, no. 15 (2002): 3326-3334; Blanquet, S., Zeijdner, E., Beyssac, E., Meunier, J. P., Denis, S., Havenaar, R., & Alric, M. (2004). A dynamic artificial gastrointestinal system for studying the behavior of orally administered drug dosage forms under various physiological conditions. Pharmaceutical research, 21(4), 585-591), or animal (see e.g. Smeets-Peeters, M., Watson, T., Minekus, M., & Havenaar, R. (1998). A review of the physiology of the canine digestive tract related to the development of in vitro systems. Nutrition research reviews, 11(1), 45-70; Leser, T. D., Amenuvor, J. Z., Jensen, T. K., Lindecrona, R. H., Boye, M., & Møller, K. (2002). Culture-independent analysis of gut bacteria: the pig gastrointestinal tract microbiota revisited. Applied and Environmental Microbiology, 68(2), 673-690).

Another possible way to control the timing and the duration of the delivery of the fecal transplant into the colon is the embedded container-based approach under electronic control described in the above-cited US Patent Application Publication No. 2010/0215732. The longer the duration of the delivery, the better the efficacy of the transplantation. Hence, dedicated containers with different cover thicknesses, can be utilized to enable different starting times for the fecal transplant to spill. This way, with multiple containers spilling contents at different times, efficacy of duration of delivery might improve. The containers can disintegrate depending on their targeted cover. The timing of when the cover will disintegrate can depend on the thickness.

Another possibility for controlling the container breakdown is by programing a microheater that can induce breakout of the container material. The microheater can be programmed in advance to change its operation state according to a set time, a set duration, or the sensing of certain features in the lumen, such as pH, or the presence/non-presence of certain bacteria concentration.

Beyond programming, another approach to register the start of the delivery is to embed in the implement a sensor or a group of sensors recognizing the disintegration of the outer shell cover of the entire implement. For example, wireless miniature moisture sensors, or impedance sensors, or pH sensors can fulfill this task. Subsequently, the delivery of the transplant can be monitored by an appropriate miniature wireless chemical sensor (see e.g. Diamond, D., Coyle, S., Scarmagnani, S., & Hayes, J. (2008). Wireless sensor networks and chemo-/biosensing. Chemical reviews, 108(2), 652.).

By detecting the disintegration of one or more components configured to disintegrate within the target organ, and communicating this to a receiver outside the body, this can be used to control the overall conveyance of the pseudobezoar through the GI tract. For example, upon detecting the arrival at the target organ, a predetermined wait time known to exceed the expected duration of the fecal transplant release time can be allowed to expire, at which time a further action to expedite the expulsion of remaining intact components from the body, as outlined herein further below under the heading “dimension control”.

### 4.4. Feedback Mechanism for Delivery Monitoring

In some situations related to the practical utilization of the proposed approach for non-invasive, transoral, organ-targeting, timed delivery of a gastrointestinal transplant in a targeted gastrointestinal organ, it would be beneficial to have a feedback mechanism to inform an external monitoring entity of the start, the progress, the dynamics (i.e. if delivery is done uniformly in the organ or via bursts), and the end of the gastrointestinal transplant delivery in the said targeted organ. Several possible feedback mechanisms are discussed herein, without them being considered limiting to other possible such mechanisms.

One such mechanism is to have at least one embedded microbiosensor within the pseudobezoar, for example, of one of the types described in Gage, D. J., Herron, P. M., Arango Pinedo, C., & Cardon, Z. G. (2008). Live reports from the soil grain—the promise and challenge of microbiosensors. Functional Ecology, 22(6), 983-989. The microbiosensor is connected to a miniature analog amplification, conditioning and wirelessly reporting microelectronic circuitry also embedded within the at least one pseudobezoar. The said microelectronic circuitry forming an integrated wireless microsystem with the microbiosensor, reports the concentration of a given bacteria typical for a healthy colon, for example, one from the family Enterobacter, to a receiving station external to the body of the patient. Examples of such microsystems are listed, for example, in Wise, K. D. (2006, December). Wireless integrated microsystems: coming breakthroughs in health care. In Electron Devices Meeting, 2006. IEDM'06. International (pp. 1-8). IEEE. In other words, in such an embodiment, the sensor is released from pseudobezoar with the fecal implant, and then monitors the effect of same on the target organ.

Another possible mechanism for feedback on the dynamics of the delivery of the at least one fecal transplant to the colon is to embed a miniature radio-frequency identification tag (RFID, for example of the type described in US Patent Application Publication 2010/0052900 (the entirety of which is incorporated herein by reference), within the bolus of the fecal transplant. In one embodiment, a monitoring receiver worn on a belt can be used to detect or estimate arrival of the pseudobezoar at the target organ. In another embodiment, ongoing position monitoring and improved accuracy of detected arrival at the target organ may be achieved by employing multiple receivers. In one such embodiment, the RFID can be followed via the triangulation operation, which is well known in the art, for example using two receivers in a belt that the patients wears, to monitor the travel of the carrier in the stomach. Also, should the dynamics of the implement's progress indicate certain blockage, for example when the implement stays in one spot for over a certain predetermined number of hours, breakout of the implement from the body can be initiated if deemed necessary.

### 4.5. Pseudobezoar Dimension Control

Pseudobezoar dimension control can be regarded as (a) a mechanism to safely expel the implement after the delivery of the gastrointestinal transplant has completed; (b) a safety mechanism to remove the implement from the body of the patient at any time; or (c) a way to optimize the delivery, the timing, the duration, or a combination thereof of the gastrointestinal transplant. Several possible means for dimension control are listed for illustrative and non-limiting purposes.

One possible approach to change the dimensions of the implement is to disintegrate it in a controlled fashion using, for example, the method described in International PCT Application Publication No. WO2013/091093, where we start with the disintegration by cutting the suture, for instance by the microheater, and then enabling the clusters to expel individually.

Another possible approach is to make the swellability of the at least one swellable cluster embedded in the at least one permeable container depend on a certain orally administrable excipient. For example, a polyacrylate-based cluster or granule would swell in high pH environment (>4), and would shrink in lower pH environment (<3). So, if the pH environment in the targeted gastrointestinal organ is made to become of higher value, the entire implement would swell. Conversely, if it is made of a lower value, it would shrink. Change in the pH in a given GI organ can be controlled by drinking specific liquids, for example basic or acidic drinks, or taking specific medications, for example antacids like Zantac.

Yet another possible way to control the dimensions of the implement is to select the number of swellable clusters and their final expanded size based on the level of their impregnation with the gastrointestinal transplant. Once the gastrointestinal transplant is sensed to be adequately delivered into the targeted gastrointestinal organ, the individual swollen clusters would selectively reduce their size, soften, or a combination thereof (for example by way of the above described pH-based control), to such extent, that the entire implement would freely pass through the entire gastrointestinal system due to natural peristalsis, and exit the said system in the manner a natural stool would.

### 4.6. Novel Aspects of the Present Invention

According to a first broad aspect of this invention, there is provided an orally administrable implement for expanding in a gastrointestinal organ of an animal, including a mammal, to deliver at least one gastrointestinal transplant to the organ, the implement including:
(a) a fluid-permeable expandable container having a first dimension and a second dimension packaged in an organ-targeting shell cover;
(b) at least one molecule cluster comprising a swellable material contained within the container and capable of swelling when contacted with a fluid;
(c) the means to integrate the implement with at least one gastrointestinal transplant obtained from a donor, or artificially synthesized, so that the said gastrointestinal transplant can be delivered in situ to a targeted gastrointestinal organ;
(d) a control mechanism to initiate and administer the delivery of the said gastrointestinal transplant in the targeted gastrointestinal organin a predetermined, or programmable, or otherwise controllable fashion;
   whereby when the implement is ingested and it reaches the targeted GI organ, the organ-targeting shell cover rapidly disintegrates, fluids from the targeted GI organ enter the fluid-permeable mesh-like container causing the molecule clusters therein to swell and the container to expand from the first dimension to the second dimension forming an intraluminal pseudobezoar, which moves inside the targeted organ as a result of natural peristalsis. In the process of its movement within the targeted gastrointestinal organ, the swollen pseudobezoar delivers the at least one gastrointestinal transplant to the said organ.

According to a second broad aspect of this invention, there is provided an orally administrable implement for expanding in a gastrointestinal organ of an animal, including a mammal, to deliver at least one gastrointestinal transplant to the organ, the implement including:
(a) a fluid-permeable expandable container having a first dimension and a second dimension packaged in an organ-targeting shell cover;
(b) at least one molecule cluster comprising a swellable material contained within the container and capable of swelling when contacted with a fluid;
(c) the means to integrate the implement with at least one gastrointestinal transplant obtained from a donor, or artificaliy synthesized, so that the said gastrointestinal transplant can be delivered in situ to a targeted gastrointestinal organ;
(d) a control mechanism to initiate and administer the delivery of the said gastrointestinal transplant in the targeted gastrointestinal organ in a predetermined, or programmable or otherwise controllable fashion;
(e) a control mechanism to change the dimensions of the entire implement on demand, when needed, or at a pre-determined moment in time, so that the said implement can exit harmlessly the gastrointestinal system.
   whereby when the implement is ingested and it reaches the targeted GI organ, the organ-targeting shell cover rapidly disintegrates, fluids from the targeted GI organ enter the fluid-permeable mesh-like container causing the molecule clusters therein to swell and the container to expand from the first dimension to the second dimension forming an intraluminal pseudobezoar, which moves inside the targeted organ as a result of natural peristalsis. In the process of its movement within the targeted gastrointestinal organ, the swollen pseudobezoar delivers the at least one gastrointestinal transplant to the said organ. Subsequently, concurrently, or at any desired moment, a control mechanism can change the dimensions of the swollen implement, so that it can exit harmlessly the gastrointestinal system.

According to a third broad aspect of this invention, there is provided an orally administrable implement for expanding in a gastrointestinal organ of an animal, including a mammal, to deliver at least one gastrointestinal transplant to the organ, the implement including:
(a) a fluid-permeable expandable container having a first dimension and a second dimension packaged in an organ-targeting shell cover;
(b) at least one molecule cluster comprising a swellable material contained within the container and capable of swelling when contacted with a fluid;
(c) the means to integrate the implement with at least one gastrointestinal transplant obtained from a donor, so that the said gastrointestinal transplant can be delivered in situ to a targeted gastrointestinal organ;
(d) a control mechanism to initiate and administer the delivery of the said gastrointestinal transplant in the targeted gastrointestinal organ of the donor in a predetermined, or programmable or otherwise controllable fashion;
(e) A feedback mechanism to report on the beginning, extent, the pattern, and the end of the delivery of the said gastrointestinal transplant in the targeted gastrointestinal organ;
(f) a control mechanism to change the dimensions of entire implement on demand, when needed, or at a pre-determined moment in time, so that the said implement can exit harmlessly the gastrointestinal system.
   whereby when the implement is ingested and it reaches the targeted GI organ, the organ-targeting shell cover rapidly disintegrates, fluids from the targeted GI organ enter the fluid-permeable mesh-like container causing the molecule clusters therein to swell and the container to expand from the first dimension to the second dimension forming an intraluminal pseudobezoar, which moves inside the targeted organ as a result of natural peristalsis. In the process of its movement within the targeted gastrointestinal organ, the swollen pseudobezoar delivers the at least one gastrointestinal transplant to the said organ. During the delivery of the gastrointestinal transplant in the targeted gastrointestinal organ, a feedback mechanism monitors and reports the extent, the pattern, and the end of the delivery of the said gastrointestinal transplant in the targeted gastrointestinal organ. Subsequently, concurrently, or at any desired moment, a control mechanism can change the dimensions of the swollen implement, so that it can exit harmlessly the gastrointestinal system.

Preferably, the implement can be self-administrable (in the case of humans) or administrable autonomously or unaided, meaning the implement is administrable in a non-invasive fashion, without the need of any external positioning or manipulating device functionally attached to it, such as an endoscope or an enema instrument.

Preferably, when the container has the first dimension, the implement can be retained in a capsule capable of being easily swallowed or administered autonomously. Once the capsule has dissolved and the container is released in the colon, the colonic fluids will enter the fluid-permeable, mesh-like, expandable container. When the fluid contacts the at least one swellable molecule cluster, the cluster will swell and the container will expand to the second dimension. The number of swellable molecule clusters in the container, their individual diameter, and their substance-delivering, liquid-retaining and liquid-absorbing properties under various pressures, as well as the design of the container itself are made such that the swollen implement has appropriate characteristics to deliver the fecal transplant material.

According to a fourth broad aspect of the invention, there is provided an orally administrable implement for expanding in a targeted gastrointestinal organ of an animal, including a mammal, to deliver at least one gastrointestinal transplant to the targeted gastrointestinal organ, the implement comprising:
(a) an organ-targeting capsule shell arranged to disintegrate in the targeted gastrointestinal organ
(b) a fluid-permeable expandable container contained within the organ-targeting capsule shell and having a first dimension, the fluid-permeable expandable container being expandable to a larger second dimension;
(c) at least one cluster comprising a swellable material contained within the container and capable of swelling when contacted with a fluid; and
(d) at least one gastrointestinal transplant carried within the organ-targeting capsule shell for in-situ delivery of the at least one gastrointestinal transplant to the targeted gastrointestinal organ;
   wherein the implement is ingestible for subsequent disintegration of the organ-targeting capsule shell inside the targeted gastrointestinal organ, whereupon fluid from the targeted gastrointestinal organ enters the fluid-permeable expandable container causing the at least one cluster therein to swell and the fluid-permeable expandable container to expand from the first dimension to the second dimension to form an intraluminal pseudobezoar which moves inside the targeted organ as a result of natural peristalsis and delivers the at least one gastrointestinal transplant to the targeted gastrointestinal organ.

The at least one gastrointestinal transplant may comprise one or more of gastrointestinal transplant material carried by the fluid-permeable expandable container, gastrointestinal transplant material carried by the swellable material inside the fluid-permeable expandable container, and gastrointestinal transplant material carried by at least one additional container that is contained within the fluid-permeable expandable container

The gastrointestinal transplant material carried by the fluid-permeable expandable container may be impregnated in a container material of the fluid-permeable expandable container, and/or woven to the fluid-permeable expandable container.

The gastrointestinal transplant material carried by the swellable material may be impregnated in the swellable material.

There may be a plurality of additional containers configured with different time-release properties by which respective deposits of transplant material in the plurality of additional containers are released in the target organ at different times.

The plurality of additional containers may vary in a wall thickness of said additional containers, and/or may comprise different respective container materials.

There may be provided a control mechanism operable to collapse the swellable material from a swollen state thereof within the targeted gastrointestinal organ.

The control mechanism may incorporate a pH-responsive collapsibility characteristic of the swellable material, and an acidic or basic ingestible fluid consumable by the animal to initiate collapse of the swellable material from the swollen state.

There may be provided a feedback mechanism operable to monitor and report progress in the delivery of the least one gastrointestinal transplant to the said targeted organ.

The feedback mechanism may comprise an ingestible transmitter contained within the fluid permeable expandable container.

In one embodiment, the ingestible transmitter is an RFID tag.

In another embodiment, the transmitter is coupled to a biosensor operable to monitor a concentration of a substance within the target gastrointestinal organ, whereby measured conditions detected by the biosensor are communicated to an external receiver.

In yet another embodiment, the transmitter is coupled to a sensor operable to detect a condition change indicative that the capsule shell has disintegrated.

According to a fifth broad aspect of the invention, there is provided a method of delivering at least one gastrointestinal transplant to a targeted gastrointestinal organ of an animal, including a mammal, the method comprising:

(a) orally administering an implement comprising an organ-targeting capsule shell, a fluid-permeable expandable container contained within the organ-targeting capsule shell and having a first dimension inside said organ-targeting capsule shell, at least one cluster contained within the container and comprising a swellable material arranged to swell when contacted with a fluid, and at least one deposit of gastrointestinal transplant material carried within the organ-targeting capsule shell;

(b) allowing the implement to reach the targeted gastrointestinal organ, whereupon the capsule shell disintegrates and fluid in said organ enters the fluid-permeable expandable container and causes the at least one cluster therein to swell and the container to expand from the first dimension to a larger second dimension; and

(c) releasing the at least one deposit of gastrointestinal transplant material into the targeted gastrointestinal organ.

Step (c) may comprise releasing a deposit of gastrointestinal transplant material from a wall of the container.

Step (b) may include forcing of the container wall against walls of the targeted gastrointestinal organ under expansion of the container from the first dimension to the larger second dimension, thereby delivering the deposit of gastrointestinal transplant material from the wall of the container to the walls of the targeted gastrointestinal organ.

Step (c) may additionally or alternatively comprise releasing at least one deposit of gastrointestinal transplant material from one or more additional containers located, at least initially, within the fluid-permeable container. The at least one deposit may be a plurality of deposits release from a plurality of additional containers at different times. The plurality of additional containers may vary from one another in wall thickness and/or wall material.

Step (c) may additionally or alternatively comprise releasing a deposit of gastrointestinal transplant material from said at least one cluster.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### 5. BRIEF DESCRIPTION OF THE DRAWINGS

The present invention, both as to its organization and manner of operation, may best be understood by reference to the following description, and the accompanying drawings of various embodiments wherein like reference numerals are used throughout the several views, and in which:

FIG. 1A is a schematic view of one embodiment of an orally administrable implement according to the invention, where the container is in the first dimension and swellable clusters are unswelled. The entire implement is encapsulated within a gelatin capsule covered with a colon-targeting Eudragit combination.

FIG. 1B is a schematic view of the orally administrable implement of FIG. 1A in the expanded second dimension as a result of swellable clusters swelling, to form a pseudobezoar. The colon-targeting capsule has already disintegrated.

FIG. 1C is a schematic view of the orally administrable implement of FIG. 1A in the expanded second dimension as a result of the swellable clusters swelling, to form a pseudobezoar. The colon-targeting capsule has already disintegrated. The container holding the swellable clusters together has started disintegrating due to chemical ageing, and the entire implement has fallen apart, with the swollen clusters becoming loose in the gastrointestinal tract.

FIG. 2A is a schematic view of one embodiment of an orally administrable implement according to the invention, where the container is in the first dimension and swellable clusters are unswelled. Within the container resides a carrier, carrying a control system and a controllable microheater, and a thread threaded through the microheater and holding the entire implement together. The implement is encapsulated within a colon-targeting gelatin capsule covered by an Eudragit combination.

FIG. 2B is a schematic view of the orally administrable implement of FIG. 2A in the expanded second dimension as a result of at least one swellable cluster swelling, to form a pseudobezoar. The carrier carrying the control system, the controllable microheater, and the thread threaded through the microheater holding the entire implement together is embedded within this pseudobezoar.

FIG. 2C is a schematic view of the orally administrable implement of FIG. 2A in the expanded second dimension as a result of the swellable clusters swelling, to form a pseudobezoar. The carrier is embedded within this pseudobezoar. The container holding swellable clusters together has started disintegrating due to the control system actuating the microheater, which in turn has melted the thread holding the pseudobezoar container together. Thus, the entire implement has fallen apart, with the swollen clusters and the carrier becoming loose in the gastrointestinal tract.

FIG. 3 shows a block-diagram of the remotely-controlled microheater-based disintegration mechanism.

### 6. DETAILED DESCRIPTION OF PREFERRED EMBODIMENT(S)

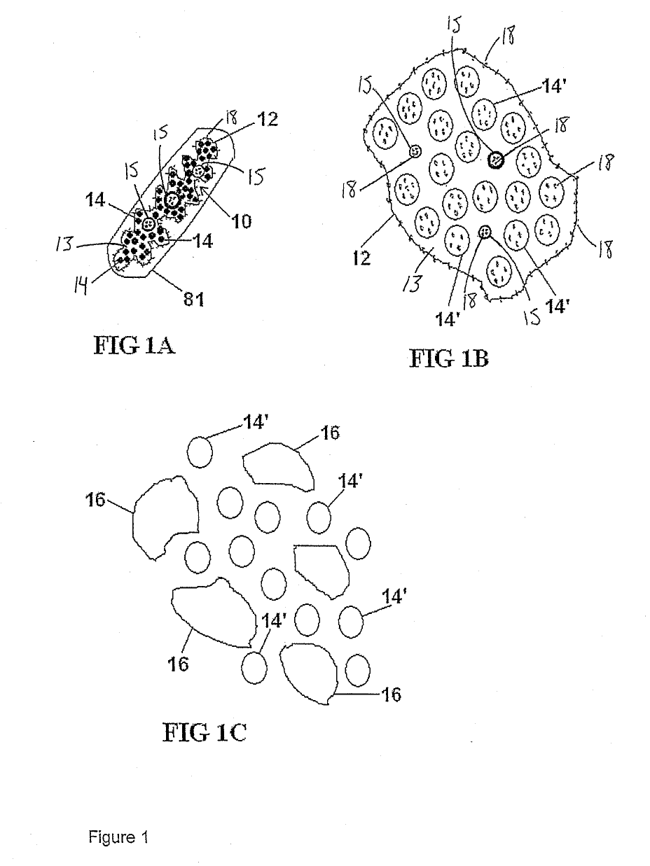

In one embodiment of the present invention, illustrated in FIGS. 1A, 1B and 1C, the orally administrable implement, referred to generally at 10, comprises biocompatible shell 81, a container 12 inside the shell and shown here in a folded, compact, first dimension. In this embodiment, the container 12 is made from a biodegradable material that allows for the passage of fluid into its interior, for example, a permeable biodegradable mesh such as Vicryl™ Knitted Mesh by Ethicon, Curacel™ by CuraMedical, or Safil™ Mesh by B Braun. Further contained in the interior 13 of container 12 is at least one cluster 14 comprising a swellable material, whereby each swellable cluster is capable of swelling when contacted with fluid, such as colonic fluid found in the colon. For example, clusters 14 can comprise Aquagel™ by Akina Inc., West Lafayette, Ind., polyacrylate, or PGX granules (Natural Factors, Vancouver, BC, Canada). In FIG. 1A, swellable clusters 14 are shown prior to contact with fluid, i.e., in their non-swelled form. In addition to clusters 14, the container 12 also contains a plurality of smaller embedded containers 15. Instead of a fluid-permeable mesh material, the embedded containers are of a biodegradable but initially fluid-impermeable material. Each of the embedded containers 15 contains fecal transplant material 18 within its initially fluid-impermeable, but biodegradable walls.

FIG. 1B shows the containers 12 of FIG. 1A in its expanded form, after the implement has been delivered into the colon, and colonic fluid has dissolved the biodegradable shell or capsule 81 and thus come into to contact with the container 12. The container 12 is now shown in its second, expanded dimension, which is of a sufficient size and shape so as to touch the walls of the colon, whereby this frictional contact with the colonic walls slows the passage of the container through the colon versus the travel time it would take to pass through the colon if the container were in its smaller unexpanded original size. The swellable clusters 14′ are now shown in their swelled state due to the colonic fluid seeping through the fluid permeable outer container 12. It is this swelling of clusters 14′ that expands container 12 to the second dimension. Preferably, the swelled clusters 14′ become spherical bodies not exceeding about 1 cm in diameter. The swellable clusters can be made of various substances, for example, appropriately cross-linked poly(acrylic acid) or poly(2-hydroxyethyl methacrylate). Preferably, the clusters are of size not permitting their exit from the container in both dry and maximally expanded states of the container, and preferably not exceeding about 1 cm when swollen in colonic fluid.

Fecal transplant material 18, whether synthetic or obtained from a donor, is applied to the swellable clusters 14 prior to incorporation thereof into the container during preparation of the implement, for example using the technique in aforementioned Takemoto et al. reference, the entirety of which is incorporated herein by reference. As a result, when colonic fluid enters the fluid-permeable outer container 12 after the shell has dissolved, the colonic fluid comes into contact with a source of fecal transplant material. On the other hand, the initially fluid-impermeable walls of the embedded containers take time to dissolve under the action of the colonic fluid, thereby delaying the exposure of the transplant material inside the embedded containers with the colonic fluid. This carrying of of transplant material by both the swellable clusters and the embedded containers increases the length of time over which transplant material is effectively dispersed into the colonic fluid versus embodiments in which the transplant material is carried by only one of these two components. As mentioned above, this longer duration of transplant material delivery may improve the efficacy of the transplantation.

As illustrated in FIGS. 1A and 1B, the wall thickness may vary among the plurality of embedded containers 15, whereby the disintegration time may vary from one embedded container to the next. This way, further staggering of the times at which different sources of the transplant material 18 are released into the colon can be effected by selecting different wall thicknesses for the embedded containers that will take different periods of exposure to the colonic fluid to dissolve. The colonic fluid will immediately be exposed to the cluster-carried deposits of transplant material once the shell has dissolved to expose the outer container 12 to the fluid. Some amount of time later, the colonic fluid will be exposed to the transplant material of the thinnest walled embedded container(s), and after a further period of time, will be exposed to the transplant material of the embedded container of next-thinnest wall material, etc. until the thickest-walled embedded container has dissolved to release its transplant material contents.

FIG. 1C represents the released pieces 16 of the container 12 in FIG. 1B once the container biodegrades after all of the transplant material has been released into the colon. Any intact pieces 16 of the container 12 are of individual size precluding the possibility of creating obstruction in the colon. The container pieces 16 along with the swelled clusters 14′ are released in the colon, so that they can be propelled out of the body by natural peristalsis in a harmless fashion.

As an alternative to use of different wall-thicknesses among a plurality of embedded containers, different container materials have different rates of disintegration in colonic fluid may be employed to stagger the release of transplant material deposits from the different embedded containers over time. In other embodiments with reduced or zero staggering of transplant material release, multiple embedded containers having the same wall material and thickness may be employed. This may include embodiments where transplant material is contained only within the embedded containers, and is not carried by the clusters 14.

As shown in FIG. 1, transplant material 18 may additionally be carried by the walls of the outer container, for example by impregnation of the transplant material into the wall material of the outer container 12, or by weaving of transplant material to the wall material of the outer container 12. Such outer-container transplant material deposits may be used in combination with embedded container transplant deposits to stagger the exposure of transplant material to the colonic fluid, whether or not the clusters 14 inside the outer container 12 also carry transplant material 18. Although the embodiment of FIG. 1 employs a plurality of swellable clusters 14 and a plurality of embedded containers, as few as one of each may be used in other embodiments. In addition, other embodiments may forgo the use of embedded containers and rely solely on carrying of transplant material by the outer container 12 and/or the one or more clusters 14. In embodiments in which transplant material is carried on an exterior of the outer container 12, the expansion of the container under the swelling of the clusters brings the transplant material into direct contact with the colonic walls.

For the sake of simplicity, some embodiments may employ one or the other, and not both, of the outer container exterior placement of transplant material and internal embedded container encapsulation of transplant material. Accordingly, one such preferred embodiment is a capsule with a container inside, which consists of a gauze material and includes multiple clusters of granules, as previously described in patent application publication 2010/0215732, with the gauze including the novel addition fecal transplant material that will come into contact with the walls of the specific organ once the granules get swollen. Another preferred embodiment has the capsule, container and granules as previously described, but includes in addition at least one dedicated container within the guaze and together with the other granules that will spill the fecal transplant material in within once the dedicated container encapsulation is dissolved. The dedicated container(s) may employ the same dissolvable material as the outer shell of the implement, or another material known that is known to dissolve under exposure to the gastrointestinal fluid of the target organ (e.g. colonic fluid, in the case that the colon is targeted).

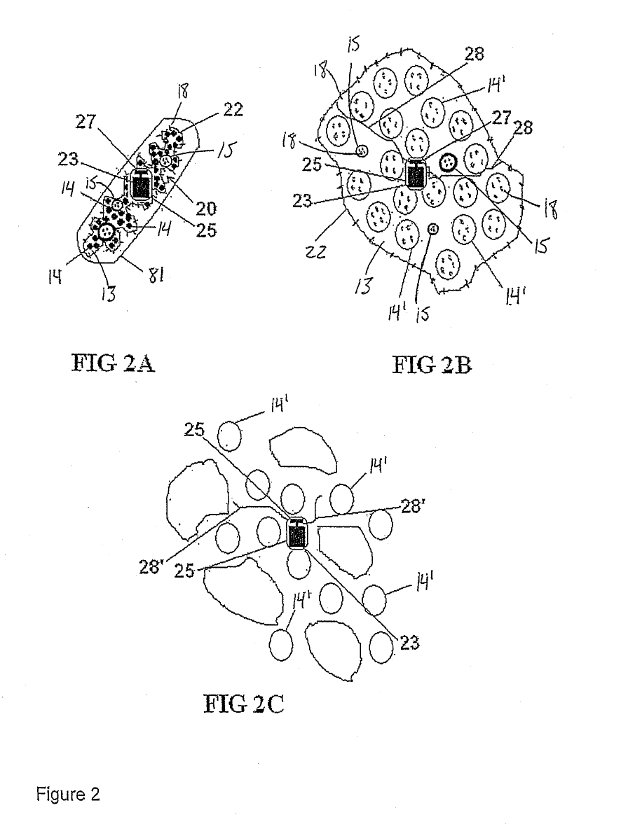

FIG. 2 shows an implement 20 of a second illustrated embodiment featuring a similar build-up of at least one pseudobezoar cluster 14, from an initial condensed or collapsed state in FIG. 2A to its swollen state, 14′, in FIG. 2B. A control system 25 is embedded within the at least one cluster, and resides in the pseudobezoar based platform within the gauze wall of the outer container 22. The control system 25 is encapsulated in an embedded biocompatible shell or carrier 23 together with a controllable microheater 27. The control system in this embodiment controls the microheater 27 that enables the disintegration of the outer container 22 which, is held together by the internal threads or sutures 28 that pass through the controllable microheater 27. The thread is stitched through multiple container wall pieces that are interconnected by this threading to form the overall container, and the travel of the thread also passes through the heater. The thread need not necessarily pass through the individual clusters of sweliable material, as they are secured within the container by the closure of the container walls around them until the time of container disintegration. As demonstrated in FIG. 2C, the thread 28′, which normally holds the container and its contents, is severed by the microheater 27 under activation of the heater by the control system 25. Once the suture is not intact, the swollen individual polymer clusters 14′, the control system 25 and the now dysfunctional microheater 27 exit the body through the GI tract in their biocompatible shell 23, just like food chime or stool would.

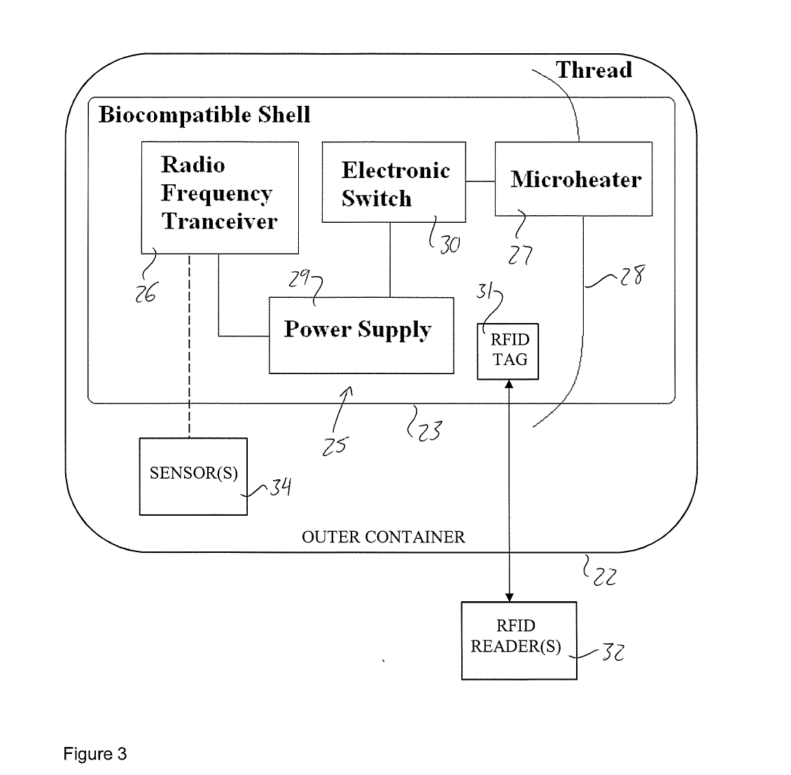

FIG. 3 shows a block-diagram of one possible implementation of a remotely-controlled system for disintegrating the entire pseudobezoar implement once the fecal transplant material has been delivered, or at an earlier time in case of possible intestinal obstruction. A radio-frequency transmitter or transceiver 26 is remotely controlled from outside the body so that a miniature microheater 27 is supplied with power from an onboard power supply 29 via an electronic switch 30 when a ‘disintegration initiation’ signal is received by the transceiver 26 from the external remote control. The microheater then melts the thread 28 that holds the entire permeable container 22 together. This control system is encapsulated in a biocompatible shell 23 and is positioned inside the permeable outer container 22. (see also FIG. 2).

For example, a miniature microheater of the type developed by Yeom et al (The design, fabrication and characterization of a silicon microheater for an integrated MEMS gas preconcentrator, J. Micromech. Microeng., 18:12 pp, 2008) can be controlled by a wireless receiver obtaining disintegration commands from the user, or from medical professional. The obtained controlling signal from the outside world turns on the embedded microheater to melt a biocompatible surgical suture holding the pseudobezoar structure together.

As opposed to wireless or remote control from outside the body, pre-programmed or user-programmable disintegration-timing options for activating the heater to initiate breakdown of the container after the transplant material has been released in the target organ could include use of a pre-programmed timer with a predetermined count-down value that is triggered to start just prior to ingestion, or a programmable timer where a user can enter a particular count-down time or select from existing count-down options just prior to ingestion. Other options could employ a programmable timer where the user enters a particular point in time at which the heater is to be activated, based on an approximation of when the implement is expected to have released all of the transplant material 18.

Still referring to FIG. 3, the shell 23 may contain an RFID tag 31 in addition to, or instead of, the control system 25 for initiating breakdown of the container. A set of RFID readers 32 in the external environment outside the patient's body, for example mounted on a belt worn by the patient, will each receive a signal from the RFID tag, from which a position of the RFID tag relative to the readers can be automatically calculated. Knowing the approximate position of the wearer's colon relative to the RFID readers can thus be used to automatically signal the patient or monitoring personnel of the arrival of the ingested implement at the colon or other targeted organ. A predetermined period of time known or estimated to be sufficient to allow full release of all the transplant material can be measured from this arrival time of the implement at the colon, and the activation signal from an external radio controller then be sent to the receiver or transceiver 26 of the control system 25 to activate the microheater 27, thereby breaking down the outer container 22 to release the swollen clusters 14′ and allow expulsion of the clusters individually from the colon. The RFID tag may be a passive RFID tag requiring no local power source within the implement, instead relying on an interrogation function of the RFID readers to transmit its ID back to the readers.

The RFID readers 32 can be used not only to confirm arrival of the implement at the target organ, but also to track its progress through the GI tract, whereby a detected lack of motion for an extended period of time prior to receipt of a successful ‘target organ arrival’ signal can be used as a signal to initiate a premature breakdown of the outer container so as to release the clusters and allow release of all components of the implement to travel through the GI tract on an expedited basis without performing the implement-slowing expansion of the outer container 22 in the target organ.

With continued reference to FIG. 3, in addition to, or instead of, detection of the arrival of the implement at the target organ by an RFID or other position-detection mechanism, one or more sensors 34 may be contained within the outer container 22 and/or in one or more embedded containers 15 in order to monitor the progress of the implement's travel and/or the progress of the transplant release process in the target organ. Miniature moisture sensors, or impedance sensors, or pH sensors inside the outer container 22, but outside the embedded container(s) 15, can be used to detect that the outer shell 81 has dissolved based on the sudden exposure of the sensor(s) 34 to the colonic fluid at such point in time. A dedicated or shared transmitter, for example the same transceiver 26 used to activate the microheater, transmits a ‘shell dissolved’ signal to an external receiver outside the body, thereby also providing a signal indicative of the implement's presence at the target organ. If one sensor is located within one of the embedded containers, a received signal from this sensor would represent a further point of progression in the overall transplant release process by marking the point in time at which the respective embedded container has dissolved.

Additionally or alternatively, the one or more sensors 34 may include a microbiosensor in the outer container 22 or an embedded container 15 that instead of detecting conditions reflective that outermost shell or embedded container has dissolved actually monitors and reports a concentration level of a substance within the colonic fluid to an external receiver to provide ongoing reporting or continual monitoring of the progress of the transplant delivery process.

As an alternative to use of the microheater to breakdown the outer container to reduce the implement down to smaller individual components, a pH responsive cluster material whose swollen size can be reversed or reduced under exposure to pH levels beyond a predetermined pH level can be used to expedite the implement's travel, either upon detected, timed or suspected completion of the transplant delivery, or in response to a detected or suspected blockage, impass, slow-down or other emergency situation in the implement's progress through the GI system. Other embodiments may employ both the microheater and pH-responsive breakdown arrangements, for example for failsafe purposes in case one such mechanism should prove ineffective to address a particular situation requiring emergency expulsion of the implement.

Since various modifications can be made in our invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. An orally administrable implement for expanding in a targeted gastrointestinal organ of an animal, including a mammal, to deliver at least one gastrointestinal transplant to the targeted gastrointestinal organ, the implement comprising:
(a) an organ-targeting capsule shell arranged to disintegrate in the targeted gastrointestinal organ
(b) a fluid-permeable expandable container contained within the organ-targeting capsule shell and having a first dimension, the fluid-permeable expandable container being expandable to a larger second dimension;
(c) at least one cluster comprising a swellable material contained within the container and capable of swelling when contacted with a fluid; and
(d) at least one gastrointestinal transplant carried within the organ-targeting capsule shell for in-situ delivery of the at least one gastrointestinal transplant to the targeted gastrointestinal organ;
wherein the implement is ingestible for subsequent disintegration of the organ-targeting capsule shell inside the targeted gastrointestinal organ, whereupon fluid from the targeted gastrointestinal organ enters the fluid-permeable expandable container causing the at least one cluster therein to swell and the fluid-permeable expandable container to expand from the first dimension to the second dimension to form an intraluminal pseudobezoar which moves inside the targeted organ as a result of natural peristalsis and delivers the at least one gastrointestinal transplant to the targeted gastrointestinal organ.

2. The implement of claim 1 wherein the at least one gastrointestinal transplant comprises gastrointestinal transplant material carried by the fluid-permeable expandable container.

3. The implement of claim 2 wherein the gastrointestinal transplant material carried by the fluid-permeable expandable container comprises impregnated gastrointestinal transplant material that is impregnated in a container material of the fluid-permeable expandable container.

4. The implement of claim 2 wherein the gastrointestinal transplant material carried by the fluid-permeable expandable container comprises woven gastrointestinal transplant material that is woven to the fluid-permeable expandable container.

5. The implement of claim 1 wherein the at least one gastrointestinal transplant comprises gastrointestinal transplant material carried by the swellable material inside the fluid-permeable expandable container.

6. The implement of claim 5 wherein the gastrointestinal transplant material carried by the swellable material is impregnated in the swellable material.

7. The implement of claim 1 wherein the at least one gastrointestinal transplant comprises gastrointestinal transplant materials carried by both the fluid-permeable expandable container and the swellable material contained therein.

8. The implement of claim 1 wherein the at least one gastrointestinal transplant comprises gastrointestinal transplant material carried by at least one additional container that is contained within the fluid-permeable expandable container.

9. The implement of claim 8 wherein the at least one additional container comprises a plurality of containers configured with different time-release properties by which respective deposits of transplant material in the plurality of containers are released in the target organ at different times.

10. The implement of claim 9 wherein the plurality of containers vary in a wall thickness of said plurality of containers.

11. The implement of claim 9 wherein the plurality of containers comprise different respective container materials.

12. The implement of claim 1 in combination with a control mechanism operable to collapse the swellable material from a swollen state thereof within the targeted gastrointestinal organ.

13. The implement of claim 12 wherein the control mechanism incorporates a pH-responsive collapsibility characteristic of the swellable material, and an acidic or basic ingestible fluid consumable by the animal to initiate collapse of the swellable material from the swollen state.

14. The implement of claim 1 wherein the swellable material has a controllable collapsibility characteristic.

15. The implement of claim 14 wherein the collapsibility characteristic is pH-responsive.

16. The implement of claim 15 in combination with an acidic or basic ingestible fluid consumable by the animal to initiate a size change of the swellable material.

17. The implement of claim 1 in combination with a feedback mechanism operable to monitor and report progress in the delivery of the least one gastrointestinal transplant to the said targeted organ.

18. The implement of claim 17 wherein the feedback mechanism comprises an ingestible transmitter contained within the fluid permeable expandable container.

19. The implement of claim 18 wherein the ingestible transmitter is an RFID tag.

20. The implement of claim 18 wherein the transmitter is coupled to a biosensor operable to monitor a concentration of a substance within the target gastrointestinal organ, whereby measured conditions detected by the biosensor are communicated to an external receiver.

21. The implement of claim 18 wherein the transmitter is coupled to a sensor operable to detect a condition change indicative that the capsule shell has disintegrated.

22. A method of delivering at least one gastrointestinal transplant to a targeted gastrointestinal organ of an animal, including a mammal, the method comprising:
(a) orally administering an implement comprising an organ-targeting capsule shell, a fluid-permeable expandable container contained within the organ-targeting capsule shell and having a first dimension inside said organ-targeting capsule shell, at least one cluster contained within the container and comprising a swellable material arranged to swell when contacted with a fluid, and at least one deposit of gastrointestinal transplant material carried within the organ-targeting capsule shell;
(b) allowing the implement to reach the targeted gastrointestinal organ, whereupon the capsule shell disintegrates and fluid in said organ enters the fluid-permeable expandable container and causes the at least one cluster therein to swell and the container to expand from the first dimension to a larger second dimension; and
(c) releasing the at least one deposit of gastrointestinal transplant material into the targeted gastrointestinal organ.

23-27. (canceled)
